(19) United States
(12) Patent Application Publication
(10) Pub. No.: US 2025/0064893 A1
(43) Pub. Date: Feb. 27, 2025

(54) METHODS FOR THE TREATMENT OF AUTOIMMUNE PULMONARY ALVEOLAR PROTEINOSIS

(71) Applicant: Savara Inc., Langhorne, PA (US)

(72) Inventor: Cecilia Ganslandt, Austin, TX (US)

(21) Appl. No.: 18/247,431

(22) PCT Filed: Sep. 30, 2021
(86) PCT No.: PCT/US2021/052911

§ 371 Date: Mar. 30, 2023

Related U.S. Application Data
(60) Provisional application No. 63/086,659, filed on Oct. 2, 2020.

(51) Int. Cl. A61K 38/19; A61K 9/00; A61P 11/00

(52) U.S. Cl. CPC A61K 38/193; A61K 9/0078; A61P 11/00

(57) ABSTRACT

Provided is a method for the treatment of moderate-severe pulmonary alveolar proteinosis (aPAP) comprising administering a formulation comprising molgramostim once daily by inhalation to a patient in need thereof.

### CROSS-REFERENCE TO RELATED APPLICATIONS

This application is the U.S. National Stage entry of International Application No. PCT/US2021/052911, filed Sep. 30, 2021, which claims the benefit of and priority to U.S. Provisional Application No. 63/086,659, filed Oct. 2, 2020, each of which are incorporated by reference in its entirety herein.

### BACKGROUND

Autoimmune pulmonary alveolar proteinosis (aPAP) is a serious, rare, and chronic debilitating pulmonary disease characterized by alveolar surfactant accumulation which results in hypoxemic respiratory failure, and innate immune deficiency. aPAP syndrome occurs in a group of heterogenous diseases that affect men, women, children, and neonates, including individuals of an ethnicities and geographic locations. aPAP affects fewer than 50,000 people worldwide (estimated 2100 US) for which whole lung lavage (WLL) is the standard of care. Whole lung lavage is an invasive treatment that is not widely available, is inefficient and associated with morbidity from general anesthesia, tracheal abrasion caused by prolonged intubation with a double lumen endotracheal tube, mechanical ventilation, and repeated filling and draining of the lung with saline while percussing the chest to emulsify surfactant lipids into the saline. Further, the procedure requires an expert bronchoscopist to perform the procedure. This procedure is invasive and more complicated and difficult in children, and is unavailable at most medical centers. Currently, there is no FDA-approved disease modifying treatment. Thus, there is a significant, unmet need for drug therapy for autoimmune aPAP.

Provided is a method for the treatment of moderate-severe pulmonary alveolar proteinosis (aPAP) comprising administering a formulation comprising 300 μg molgramostim once daily by inhalation to a patient in need thereof.

These and other objects of the invention are described in the following paragraphs. These objects should not be deemed to narrow the scope of the invention.

### BRIEF DESCRIPTION OF THE DRAWINGS

FIG. 1 shows the change from baseline in the alveolar-arterial difference in oxygen concentration at week 24 in the continuous-molgramostim group (46 patients) and the placebo group (47 patients). The analysis was performed with the use of the revised full analysis set, in which invalid data for 1 patient in each molgramostim group and 2 patients in the placebo group were treated as missing data and were replaced by means of multiple imputation (indicated by an x). Each circle represents the result for 1 patient. The horizontal lines represent means, and the I bars standard errors.

FIG. 2 shows the mean change from baseline during the blinded intervention period and the open-label treatment-extension period in the continuous-molgramostim group (46 patients) and the placebo group (47 patients) for the following end points: the alveolar-arterial difference in oxygen concentration (A-aD_{O2}) (Panel A), the percent of predicted diffusing capacity of the lung for carbon monoxide (DL_{CO}) (Panel B), the total score on the St. George's Respiratory Questionnaire (SGRQ; scores range from 0 to 100, with higher scores indicating more severe effects on functional health status) (Panel C), and the distance covered on the 6-minute walk test (Panel D). The patients received the assigned intervention from week O to week 24 and then received intermittent molgramostim from week 24 to week 72. Data missing during the blinded intervention period were replaced by means of multiple imputation. The difference between the number of patients with data for weeks 24 through 48 and the number with data for weeks 48 through 72 is due to a protocol amendment that permitted the use of a longer open label treatment-extension period for some patients. T bars indicate standard errors.

FIG. 3 shows change in A-aD_{O2} from Baseline to Week 24 using the Full Analysis Set and Outlier Analysis of the Results of Arterial Blood Gas Measurements. Patients breathing room air during specimen collection for arterial blood gas analysis are indicated by green circles (continuous molgramostim; Panels A, C, D) or grey circles (placebo Panels A, E) or open circles (either molgramostim or placebo; Panels B, F) while those breathing supplemental oxygen via nasal canula at various rates are indicated by orange circles (continuous molgramostim, n=1; Panels A, B, C, F), Purple or salmon circles (Panel A, B, E, F), or blue circles (Intermittent molgramostim (n=1; Panels B, D, F). Panel A shows the mean (±SEM) change from baseline to week 24 in A-aD_{O2} in the continuous molgramostim group (46 patients) compared to the placebo group (47 patients) during the randomized double-blind intervention period analyzed using the FAS. Panel B shows the relationship between PaO2 and A-aD_{O2} results for all arterial blood gas measurements (n=541) in all enrolled study patients (n=138) including patients breathing room air (open circles) or supplemental oxygen via nasal canula (colored circles) during the collection of blood specimens for analysis. Each circle represents one measurement in one patient. Each color represents a different patient. Panels C-E show quantile-quantile plots of the distribution of A-aD_{O2} results (Normal Data Quantiles) compared to a normal distribution (Normal Theoretical Quantiles) for results at baseline (Visit 2) and week 24 (Visit 8) for patients in the continuous molgramostim group (n=46, Panel C), intermittent molgramostim group (n=45, Panel D), and placebo group (n=47, Panel E). Panel F shows robust nonlinear regression outlier analysis of the A-aD_{O2} results for all patients in the indicated groups.

FIG. 4 shows the sensitivity Analysis for the Primary Endpoint During the Double-Blind Period. Least squares mean (±95% confidence intervals) values are shown for each sensitivity analysis for the comparison of the change from baseline at 24 weeks in AaD_{O2} between the continuous molgramostim group (46 patients) and placebo group (47 patients) performed using the R-FAS.

FIG. 5 shows responder analysis for the comparison of SGRQ total scores in patients receiving molgramostim versus placebo during the double-blind period. Panel A shows odds ratio and 95% confidence interval (95% CI) for a response for the patients receiving continuous molgramostim (n=46) or placebo (n=47) at the indicated response threshold levels (4, 8, or 12 points) of the SGRQ total score. Panel B shows similar analysis of the results for patients recieving intermittent molgramostim (n=45) compared to placebo (n=47).

FIG. 6 shows a Comparison of the Prior use of Whole Lung Lavage Therapy Among the Three Intervention Groups. Panel A shows a Kaplan-Meier plot of the percentage of the 138 patients in the three randomized intervention groups who underwent whole lung lavage prior to enrollment in this study.

FIG. 7 shows an analysis of the use of Whole Lung Lavage Therapy Before and During the IMP ALA Trial. Panel A shows the hazard ratio for the time to first use of WLL and rate ratio for the freuency of WLL use in patients receiving continuous molgramostim or intermittent molgramostim compared to placebo during the double-blind period. Panel B shows the rate of WLL use, expressed in number of single-lung WLL procedures per patient year before randomization in the IMPALA trial (Pre-trial), in the patients in receiving continuous molgramostim (n=46), intermittent molgramostim (n=45), or placebo (n=47) during the randomized treatment period (double-blind period), and in the patients (n=131) enrolled in the open-label treatment extension period (Open-label period). Data analyzed using the FAS.

FIG. 8 shows the change in Serum Biomarker Levels During the Double-Blind Intervention Period. Shown are mean (±SEM) values at Baseline (A) and mean (±SEM) percentage change from baseline at 24 weeks for serum biomarkers of aPAP including lactate dehydrogenase (LDH), Krebs von den Lungen-6 (KL-6), carcinoembryonic antigen (CEA), cytokeratin fragment 19 (Cyfra 21-1), surfactant protein A (SP-A), surfactant protein B (SPB), surfactant protein C (SP-C), and surfactant protein D (SP-D) in the patients receiving continuous molgramostim, intermittent molgramostim, or placebo (indicated). Analysis was performed on the FAS of observed values without imputation of missing data.

FIG. 9 shows change Over Time in Blood Hematocrit and Hemoglobin Levels in Patients Receiving Continuous Molgramostim or Placebo During the IMPALA Trial. Panel A shows the mean (±SEM) hematocrit in patients receiving continuous molgramostim (n=46) or placebo (n=47) during randomized treatment period (Blinded period) or open-label extension treatment period (n=131) (Open-label period). Data are shown for the FAS of observed values without imputation of missing data.

FIG. 10 shows changes in A-aD_{O2}, DL_{CO}%, SGRQ Scores, and GGO Scores in Patients Receiving Molgramostim or Placebo During the Double-Blind Treatment Period. Results are mean (±SEM) changes for each endpoint in patients receiving intermittent molgramostim (green bars) vs placebo (gray bars); for comparison results for patients receiving continuous molgramostim are shown (open bars). Panel A shows the results for changes in the alveolar-arterial difference in oxygen concentration (A-aD_{O2}). Panel B shows the results for changes in DL_{CO}%. Panel C shows the results for SGRQ total score, and activity, impact, and symptom components. Panel D shows the results for chest CT GGO scores at baseline (Visit 2), week 24 (Visit 8), and the change from baseline to week 24. Panel A was analyzed using the R-FAS and Panels B-D with the FAS with imputation of missing data except for the SGRQ component scores, which include only observed values without immutation.

FIG. 11 shows changes from Baseline Over Time in A A-aD_{O2}, DL_{CO}, SGRQ total score, and 6MWT-distance in Patients Receiving Intermittent Molgramostim or Placebo During the Double-Blind and Open-Label Treatment Periods. Panels A-D show the mean (±SEM) changes from baseline to week 24 (Double-blind Period, white regions) and from week 24 to week 72 (Open-label Period, grey regions) for A-aD_{O2} (Panel A), DL_{CO}% (Panel B), SGRQ total score (Panel C), and the distance covered in a six-minute walk test analyzed using the R-FAS (Panel A) or FAS (Panels B-D). Panel A was analyzed using the R-FAS and Panels B-D with the FAS with imputation of missing data.

### DETAILED DESCRIPTION

This detailed description is intended only to acquaint others skilled in the art with the present invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this patent application, and may be variously modified.

As used in the specification and the appended claims, unless specified to the contrary, the following terms have the meaning indicated:

The term “moderate-severe puirnonary alveolar proteinosis” refers to an alveolar-arterial difference in oxygen tension (A-aD_{O2}) of 40.6±18.0 mmHg and disease severity score (DSS) of 2.9±1.1 (data are mean ±SD).

The term “pharmaceutically acceptable” is used adjectivally to mean that the modified noun is appropriate for use as a pharmaceutical product for human use or as a part of a pharmaceutical product for human use.

The term “subject” includes humans and other primates as well as other mammals. In some embodiments, the subject is a human.

The term “therapeutically effective amount” means a sufficient amount of the API or pharmaceutical composition to treat a condition, disorder, or disease, at a reasonable benefit/risk ratio applicable to any medical treatment.

The terms “treat”, “treating” and “treatment” refer to a method of alleviating or abrogating a condition, disorder, or disease and/or the attendant symptoms thereof.

As used herein, “about” means ±20% of the stated value, and includes more specifically values of ±10%, ±5%, ±2% and ±1% of the stated value.

The methods described herein provide for the administration of recombinant human GM-CSP (molgramostim) by inhalation. Molgramostim is produced by using recombinant DNA technology in an Escherichia coli expression system, which results in production of non-glycosylated recombinant human GM-CSP. Structurally, molgramostim is a compact globular protein containing a four-helix bundle and a closely packed hydrophobic core. X-ray crystal structure of molgramostim indicate a-helical and P-sheet content of 40-50% and 6-10%, respectively. See, e.g., Walter et al. J. Mol. Biol. 1992; 224:1075-85, which is incorporated herein by reference for all purposes.

Molgradex is an inhaled formulation of recombinant human granulocyte-macrophage colony-stimulating factor (GM-CSP). The formulation comprises recombinant human albumin as a stabilizer. In some embodiments, the stabilizer is Recombumin. The formulation further comprises a buffer at pH 7.0 to 7.4. In some embodiments, the buffer is a citrate phosphate buffer. In some embodiments, the formulation contains 250 μg/mL molgramostim in 1.2 mL solution.

In some embodiments, the formulation is delivered via a nebulizer, a high-efficiency nebulizer, or a dry-powder inhaler.

In some embodiments, the nebulizer is a jet nebulizer, an ultrasonic nebulizer, a pulsating membrane nebulizer, a nebulizer comprising a vibrating mesh or plate with multiple apertures, a nebulizer comprising a vibration generator and an aqueous chamber, or a nebulizer that uses controlled device features to assist inspiratory flow of the aerosolized aqueous solution.

In some embodiments, the nebulizer is a vibrating mesh nebulizer, such as a PARI eFlow Nebulizer System (PARI Pharma GmbH, Germany). The eFlow Nebulizer is a single patient use, reusable electronic nebulizer. It includes a fine particle aerosol generator (perforated vibrating membrane) defined by a 30L mesh and an aerosol chamber that can produce aerosols with high density of active drug, precisely defined droplet size and a high proportion of respirable droplets.

In some embodiments, the molgramostim is administered continuously. In some embodiments, the molgramostim is administered continuously intermittently (e.g., every other week).

The pharmaceutical formulations, methods, and uses described herein will be better understood by reference to the following exemplary embodiments and examples, which are included as an illustration of and not a limitation upon the scope of the invention.

### EXAMPLES

### Example 1: IMPALA Trial

IMPALA is a Phase III, double-blind, placebo-controlled, randomized trial of inhaled recombinant human GM-CSF (molgramostim) in patients with moderate-severe autoimmune pulmonary alveolar proteinosis (APAP). 138 patients were randomized and received treatment for 24 weeks in one of three arms: 1) Molgradex 300 μg administered once daily continuously over 24 weeks, 2) Molgradex 300 μg, and matching placebo, administered once daily in 7-day intermittent cycles of each, or 3) inhaled placebo administered once daily continuously over 24 weeks. Compared to other cohorts, IMPALA patients have relatively severe disease as demonstrated by an alveolar-arterial difference in oxygen tension (A-aD_{O2}) of 40.6±18.0 mmHg and disease severity score (DSS) of 2.9±1.1 (data are mean ±SD). Whereas most series report a 2:1 male predominance, 43% of subjects in IMP ALA are female.

The primary end point was the mean change from baseline in the A-aD_{O2} at week 24. Key secondary end points that informed direct patient benefit included the mean change from baseline to week 24 in functional health status, measured with the total score on the St. George's Respiratory Questionnaire (SGRQ; scores range from Oto 100, with higher scores indicating more severe effects on functional health status); the mean change from baseline to week 24 in the distance covered on the 6-minute walk test; and the time from randomization to the first use of whole lung lavage. Other end points included the mean change from baseline to week 24 in the DL_{CO}, SGRQ component scores (activity, impact, and symptoms), number of whole-lung lavage procedures, forced expiratory volume in 1 second, forced vital capacity, vital capacity, score for ground glass opacification on chest CT (scores range from Oto 15, with higher scores indicating a higher proportion of the area of the lung parenchyma on a CT scan corresponding to regions affected by ground-glass opacification, an indication of the abnormal accumulation of surfactant sediment in patients with aPAP), and serum biomarker levels. These end points permitted evaluation of a range of abnormalities that are driven by the pathogenesis of PAP. Safety was assessed by monitoring adverse events.

The trial was initially powered to detect a difference of 10 mm Hg between the molgramostim groups and the placebo group in the mean change in the A-aD_{O2}. The trial was expanded to include several outcome measures (change in the SGRQ total score, change in the distance covered on the 6-minute walk test, and time from baseline to the first use of whole-lung lavage) were promoted as key secondary end points, and the sample size was recalculated to give the trial 90% power at the 5% significance level to detect a difference of 50 m between the continuous-molgramostim group and the placebo group in the mean change in the distance covered on the 6-minute walk test.

As prespecified in the statistical analysis plan the primary end point was evaluated with the use of an analysis of covariance model that included trial group, status with regard to whole-lung lavage within 2 months before baseline (receipt vs. nonreceipt), and geographic region (Japan vs. other countries) as factors and baseline values as covariates; a P value of less than 0.05, on the basis of a comparison of least-squares means, was considered to indicate statistical significance. To control for type I error, key secondary end points were analyzed with the use of a testing hierarchy in which the continuous-molgramostim and placebo groups were compared first, and if significance was reached in the evaluation of any key secondary end points, then comparison of the intermittent-molgramostim and placebo groups would proceed, first with evaluation of the primary end point, and if significance was reached, then with evaluation of the key secondary end points. The threshold indicating significance for analyses of key secondary end points was adjusted for multiplicity with the use of the truncated Hochberg procedure. Analyses of all other end points were considered to be supportive and were not adjusted for multiplicity, and P values are not reported for these end points. The full analysis set, which included results for all patients who received at least one dose of the assigned intervention was used for the initial analysis of all data. After trial data were unblinded and the prespecified analysis was conducted, a nonphysiologic (large negative) A-aD_{O2} value (−42 mm Hg) was identified in one patient. A reexamination of all relevant data revealed that this patient and three others (one in the continuous-molgramostim group, one in the intermittent-molgramostim group, and two in the placebo group) had undergone blood gas analysis while they were receiving supplemental oxygen through a nasal cannula, which precluded calculation of the A-aD_{O2} because the fraction of inspired oxygen was unknown. Therefore, a revised full analysis set was established in which the invalid blood gas results for these four patients were treated as missing data, with replacement by means of multiple imputation; the revised set was used for an intention-to-treat analysis of the primary end point. Although not prespecified, for consistency, missing data for the primary end point, key secondary end points, and ground glass opacification (but not for other end points) during the blinded intervention period were replaced by means of multiple imputation. The full analysis set was used for analysis of all other end points. Results are presented as means ±SD, and analyses were performed with the use of SAS software, version 9.4 (SAS Institute).

A total of 235 patients underwent assessment for eligibility, and 138 patients with aPAP underwent randomization; 46 were assigned to receive continuous molgramostim, 45 to receive intermittent molgramostim, and 47 to receive placebo. Of these patients, 98% in each molgramostim group and 94% in the placebo group completed the blinded intervention period, and 131 were enrolled in the open-label treatment-extension period. One third of the patients received supplemental oxygen therapy during the trial. The baseline characteristics of the patients were similar in the three groups (Table 1 and Table S2).

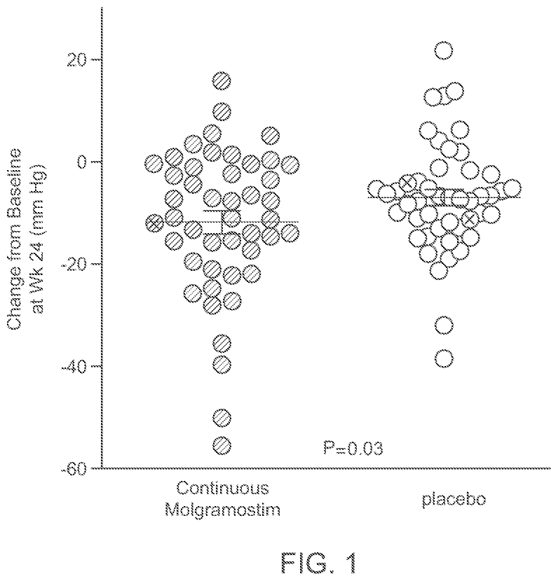

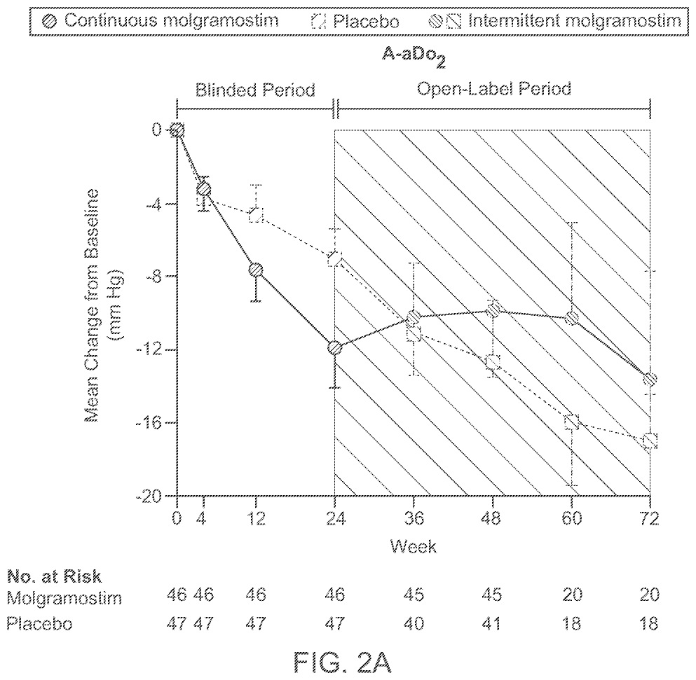

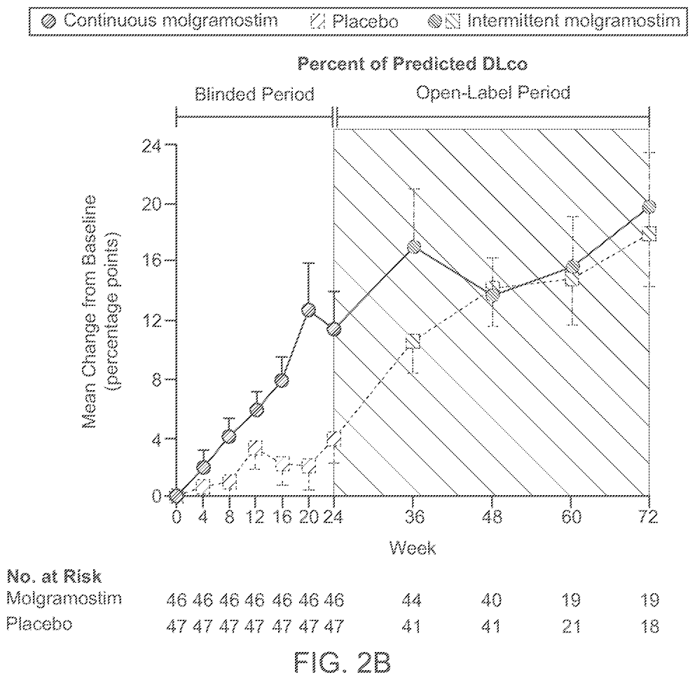

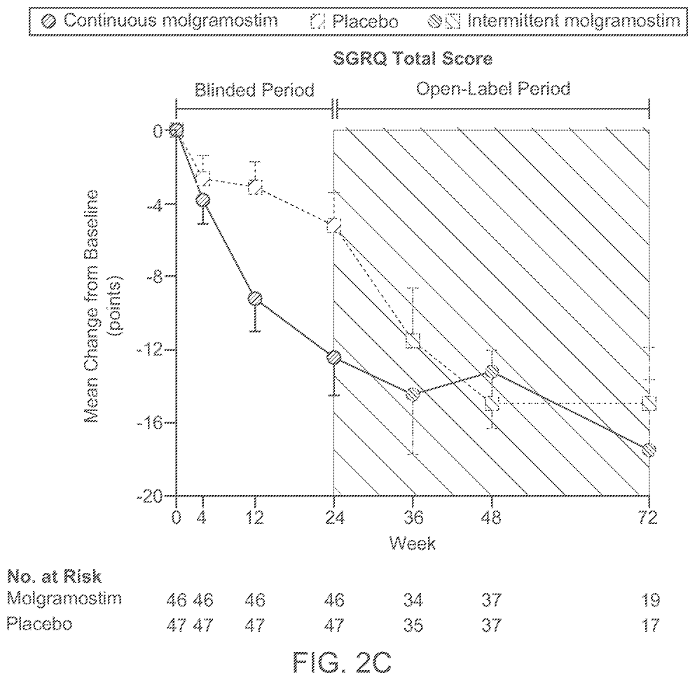

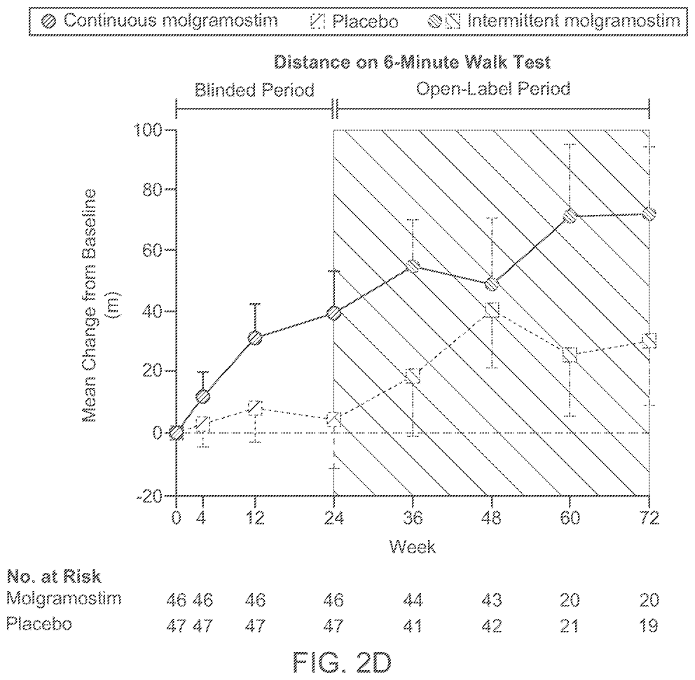

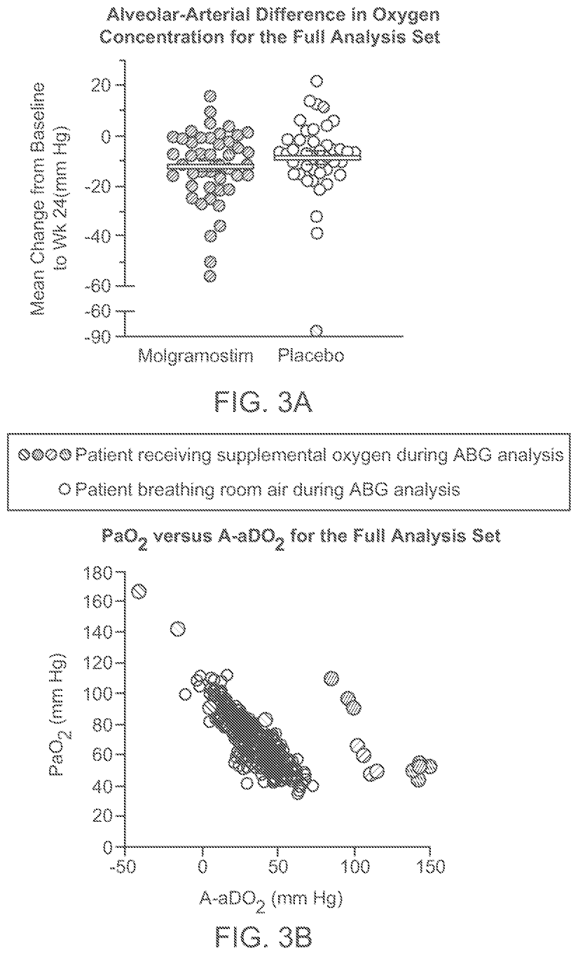

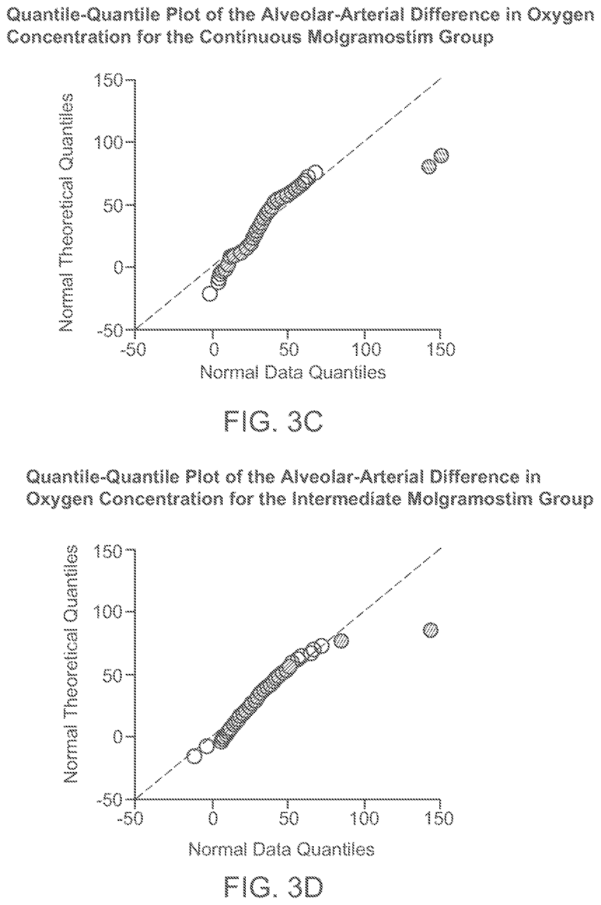

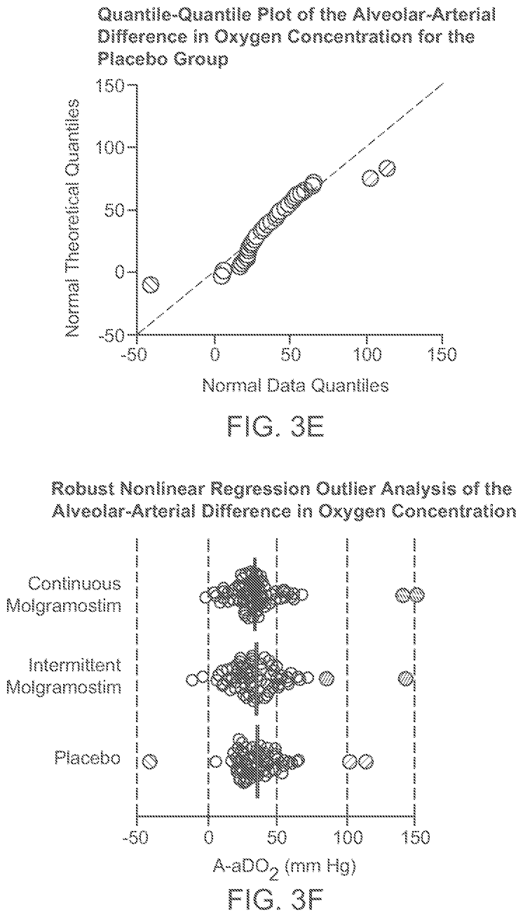

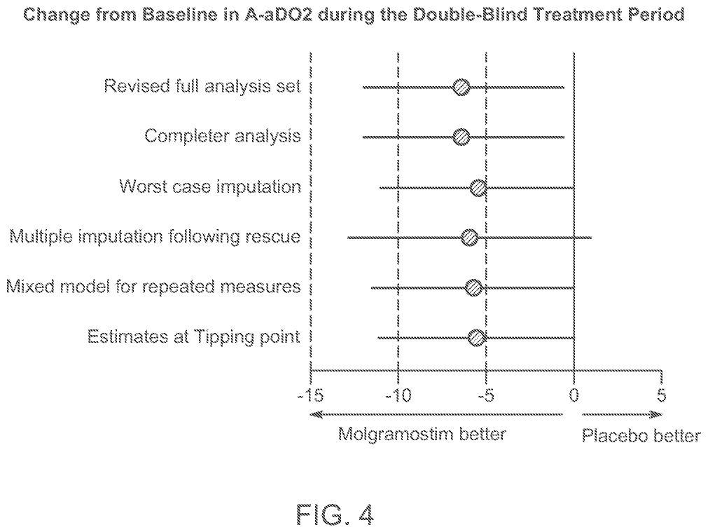

The primary end point-the mean change from baseline in the A-aD_{O2} at week 24-was not significantly different between the continuous molgramostim group and the placebo group when analyzed with the use of the full analysis set (−12.7 mm Hg vs. −7.6 mm Hg; estimated treatment difference, −5.2 mm Hg; P=0.12 by comparison of least-squares means). However, after the invalid data for patients who received supplemental oxygen during blood gas measurement were replaced by means of imputation, the change was greater among patients receiving continuous molgramostim than among those receiving placebo (−12.8 mm Hg vs. −6.6 mm Hg; estimated treatment difference, −6.2 mm Hg; P=0.03 by comparison of least-squares means) (FIGS. 1 and 2, Table 2, FIG. 3). The greater improvement in the A-aD_{O2} with continuous molgramostim was supported by results for another prespecified measure of pulmonary gas transfer, the mean change from baseline in the percent of predicted DL_{CO} at week 24, which was greater in the continuous molgramostim group than in the placebo group (12.0 vs. 4.2 percentage points; estimated treatment difference, 7.8 percentage points; 95% confidence interval [CI], 2.3 to 13.3 by comparison of least-squares means) (FIG. 2, Table 2), and was also supported by results of sensitivity analyses (FIG. 4).

TABLE 1
Selected Clinical Characteristics of the Patients at Baseline.*

| | Continuous | Intermittent | |
|---|---|---|---|
| | Molgram- | Molgram- | |
| | ostim | ostim | Placebo |
| Characteristic | (N = 46) | (N = 45) | (N = 47) |
| Age - yr | 54.0 ± 13.3 | 49.2 ± 14.1 | 46.1 ± 14.8 |
| Female sex - no. (%) | 18 (39) | 19 (42) | 22 (47) |
| Pulmonary gas transfer | | | |
| A-aDO₂ - mm Hg† | 40.5 ± 19.6 | 40.9 ± 20.2 | 40.2 ± 14.3 |
| DLco - % of | 51.9 ± 18.5 | 46.1 ± 14.5 | 49.6 ± 14.3 |
| predicted value | | | |
| Radiologic evaluation | | | |
| of the lungs | | | |
| Ground-glass opacification | 10.9 ± 3.2 | 10.8 ± 3.0 | 10.9 ± 2.8 |
| score - points‡ | | | |
| Functional health status | | | |
| SGRQ total score - points§ | 47.2 ± 20.4 | 44.4 ± 21.4 | 44.1 ± 21.7 |
| Distance on 6-minute | 411 ± 143 | 447 ± 117 | 447 ± 125 |
| walk test - m | | | |
| Previous therapy for PAP | | | |
| Whole-lung lavage therapy | | | |
| Any previous use - no. | 23 (50) | 31 (69) | 30 (64) |
| of patients (%) | | | |
| Mean no. of previous | 3.8 | 3.7 | 2.8 |
| procedures per patient | | | |
| Time since last | 24.3 ± 52.6 | 18.9 ± 24.0 | 17.7 ± 20.7 |
| procedure - mo | | | |
| GM-CSF therapy | | | |
| Any previous use - no. | 6 (13) | 7 (16) | 6 (13) |
| of patients (%) | | | |
| Time since last | 35.4 ± 35.9 | 37.8 ± 26.4 | 18.3 ± 22.6 |
| administration - mo | | | |

*Plus-minus values are means ± SD. Details regarding additional clinical characteristics of the patients at baseline are provided in Table S2. A-aDO₂ denotes alveolar-arterial difference in oxygen concentration, DLco diffusing capacity of the lung for carbon monoxide, GM-CSF granulocyte-macrophage colony-stimulating factor, PAP pulmonary alveolar proteinosis, and SGRQ St. George's Respiratory Questionnaire.
†Values were calculated with the use of the following equation: A-aDO₂ = [Fio₂ × (PB − P_{H20}) − Paco₂/R] − Pao₂. The term Fio₂ denotes fraction of inspired oxygen (assumed to be 0.21 in patients breathing ambient air), Paco₂ partial pressure of arterial carbon dioxide, Pao₂ partial pressure of arterial oxygen, PB barometric pressure (measured by validated barometers), P_{H20} partial pressure of water vapor in alveolar air (assumed to be 47 mm Hg), and R respiratory quotient (assumed to be 0.8). The analysis was performed with the use of the revised full analysis set, in which invalid A-aDO₂ data for four patients (one in each molgramostim group and two in the placebo group) were treated as missing data and were replaced by means of multiple imputation.
‡Ground-glass opacification scores range from 0 to 15, with higher scores indicating a higher proportion of the area of the lung parenchyma on a computed tomographic scan corresponding to regions affected by ground-glass opacification, an indication of the abnormal accumulation of surfactant sediment in patients with aPAP. Data for one patient (in the intermittent-molgramostim group) were unavailable and were not imputed.
§SGRQ total scores range from 0 to 100, with higher scores indicating more severe effects on functional health status.

TABLE S2
Clinical Characteristics of the Patients at Baseline.*

| | Continuous | | Intermittent | | | |
|---|---|---|---|---|---|---|
| | Molgram- | | Molgram- | | Placebo | |
| | ostim | | ostim | | Group | |
| Characteristic | (N = 46) | | (N = 45) | | (N = 47) | |
| Age - yr | 54.0 ± 13.3 | | 49.2 ± 14.1 | | 46.1 ± 14.8 | |
| Female gender - no. (%) | 18 | (39.1) | 19 | (42.2) | 22 | (46.8) |
| Tobacco use - no. (%) | | | | | | |
| Never smoker | 13 | (28.3) | 16 | (35.5) | 16 | (34.0) |
| Ex-smoker | 27 | (58.7) | 20 | (44.4) | 20 | (42.6) |
| Current smoker | 6 | (13.0) | 9 | (20.0) | 11 | (23.4) |
| Clinical and laboratory | | | | | | |
| features | | | | | | |
| Time since diagnosis of | 39.8 ± 58.1 | | 40.0 ± 45.9 | | 32.0 ± 31.5 | |
| PAP - months | | | | | | |
| Serum GM-CSF | 68,561 ± 113,710 | | 45,364 ± 54,474 | | 66,756 ± 110,907 | |
| autoantibody titer † | | | | | | |
| Hematocrit - % | 46.9 ± 5.2 | | 48.8 ± 5.7 | | 48.2 ± 5.7 | |
| Hemoglobin - g/dL | 15.1 ± 1.73 | | 15.5 ± 1.78 | | 15.3 ± 1.86 | |
| Pulmonary gas exchange | | | | | | |
| A-aDO₂ | | | | | | |
| No. of patients with | 45 | | 44 | | 45 | |
| data [R-FAS §] | | | | | | |
| Mean value - mm HG ‡ | 38.1 ± 10.8 | | 38.6 ± 12.9 | | 38.8 ± 11.2 | |
| PaO₂ | | | | | | |
| No. of patients with | 45 | | 44 | | 45 | |
| data [R-FAS §] | | | | | | |
| Mean value - mm HG ‡ | 65.5 ± 9.9 | | 66.0 ± 11.4 | | 67.1 ± 13.2 | |
| Percent of predicted | 52.1 ± 18.6 | | 46.1 ± 14.5 | | 49.6 ± 14.3 | |
| DLCO | | | | | | |
| Pulmonary airflow | | | | | | |
| and lung values | | | | | | |
| Percent of predicted FEV₁ | 89.3 ± 23.7 | | 82.5 ± 22.4 | | 79.3 ± 17.7 | |
| Percent of predicted FVC | 83.0 ± 21.5 | | 80.4 ± 12.2 | | 80.2 ± 16.7 | |
| Percent of predicted VC | 78.6 ± 3.2 | | 74.8 ± 19.5 | | 74.1 ± 18.6 | |
| Disease severity score | | | | | | |
| (DSS) - no. (%) ¶ | | | | | | |
| DSS-1 | 4 | (8.7) | 5 | (11.1) | 3 | (6.4) |
| DSS-2 | 12 | (26.1) | 14 | (31.1) | 16 | (34.0) |
| DSS-3 | 17 | (37.0) | 13 | (28.9) | 14 | (29.8) |
| DSS-4 | 5 | (10.9) | 9 | (20.0) | 10 | (21.3) |
| DSS-5 | 8 | (17.4) | 3 | (6.7) | 4 | (8.5) |
| Pulmonary surfactant | | | | | | |
| accumulation | | | | | | |
| No. of patients with data | 46 | | 43 | | 47 | |
| CT GGO score \|\| | 10.9 ± 3.2 | | 10.8 ± 3.0 | | 10.9 ± 2.8 | |
| Functional health status | | | | | | |
| Saint George Respiratory | | | | | | |
| Questionnaire ** | | | | | | |
| Total score | 47.2 ± 20.4 | | 44.4 ± 21.4 | | 44.1 ± 21.7 | |
| Activity domain score | 60.7 ± 21.5 | | 57.2 ± 25.4 | | 55.6 ± 22.6 | |
| Impact domain score | 38.9 ± 23.6 | | 35.3 ± 22.3 | | 35.8 ± 23.5 | |
| Symptom domain score | 46.3 ± 22.7 | | 47.1 ± 25.4 | | 47.1 ± 24.3 | |
| Exercise capacity | | | | | | |
| No. of patients with data | 45 | | 45 | | 47 | |
| Distance walked on | 412 ± 144 | | 447 ± 117 | | 447 ± 125 | |
| 6-min walk test (m) | | | | | | |
| Prior or concomitant | | | | | | |
| therapy of PAP | | | | | | |
| Supplemental | 15 | (32.6) | 12 | (26.7) | 12 | (25.5) |
| oxygen - no. (%) | | | | | | |
| WLL therapy ^{††} | | | | | | |
| Any prior WLL - no. | 23 | (50.0) | 31 | (68.9) | 30 | (63.8) |
| of patients (%) | | | | | | |
| No. of prior | 3.8 | | 3.7 | | 2.8 | |
| WLL procedure | | | | | | |
| Time since last WLL | 24.3 ± 52.6 | | 18.9 ± 24.0 | | 17.7 ± 20.7 | |
| procedure - mo | | | | | | |
| GM-CSF therapy | | | | | | |
| Any prior GM-CSF - no. | 6 | (13.0) | 7 | (15.6) | 6 | (12.8) |
| of patients (%) | | | | | | |

| | | | |
|---|---|---|---|
| Time since last | 35.4 ± 35.9 | 37.8 ± 26.4 | 18.3 ± 22.6 |
| administration - mo | | | |

*Plus-minus values are means ± SD. Results represent data for N = 46, 45, or 47 patients in the Continuous Molgramostim, Intermittent Molgramostim, and placebo groups, respectively, unless indicated otherwise; data include observed values only without any imputation of missing or invalid data. A-aDO₂ denotes alveolar-arterial oxygen difference, CT computed tomography, DLCO diffusing capacity for carbon monoxide, DSS [PAP] disease severity score, FEV₁ forced expiratory volume in 1 second, FVC forced vital capacity, GGO ground glass opacification score, GM-CSF granulocyte/macrophage-colony stimulating factor, R-FAS revised full analysis set, VC vital capacity, WLL whole lung lavage.

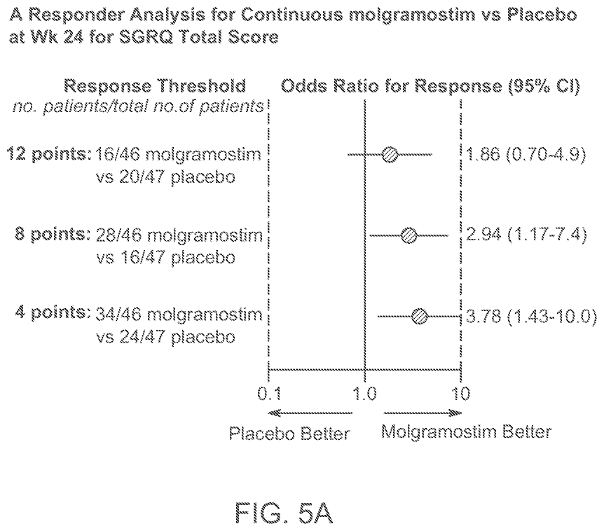

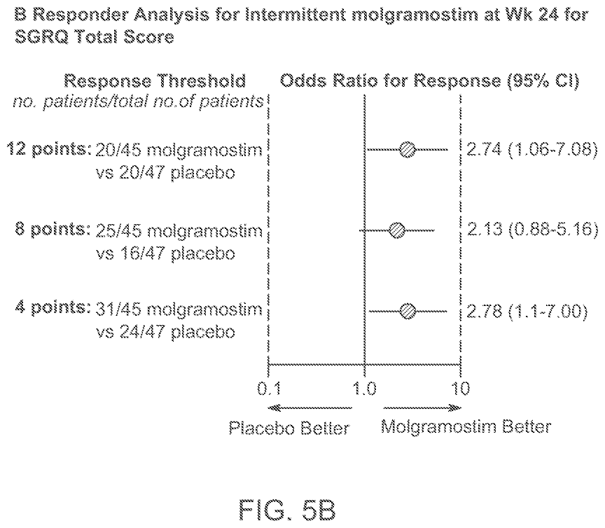

The mean change from baseline in the SGRQ total score at week 24 was significantly greater in the continuous-molgramostim group than in the placebo group (−12.4 points vs. −5.1 points; estimated treatment difference, −7.4 points; 95% CI, −13.1 to −1.6; P=0.01 by comparison of least squares means) (FIG. 2 and Table 2), as was the mean change in the SGRQ component score for activity and for impact but not for symptoms. The odds ratios for response at the 4-point, 8-point, and 12-point thresholds for change in the SGRQ total score among patients receiving continuous molgramostim as compared with those receiving placebo were 3.8 (95% CI, 1.4 to 10.0), 2.9 (95% CI, 1.2 to 7.4), and 1.9 (95% CI, 0.7 to 4.9), respectively (FIG. 5). The mean change from baseline in the distance covered on the 6-minute walk test at week 24 was not significantly different between the continuous-molgramostim group and the placebo group (32.4 m vs. 7.9 m; estimated treatment difference, 24.6 m; 95% CI,-15.3 to 64.4 by comparison of least-squares means) (FIG. 2 and Table 2).

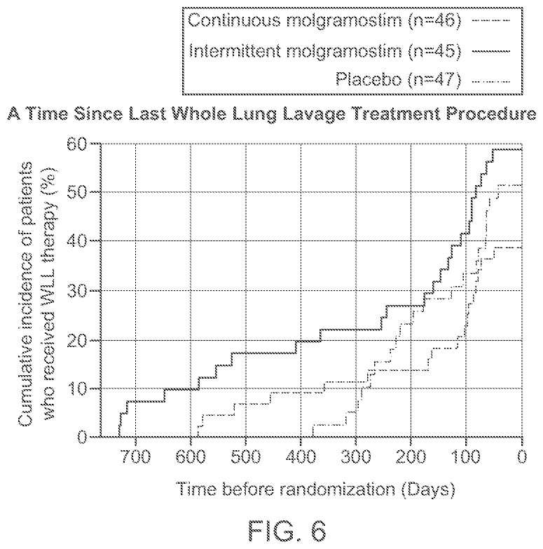

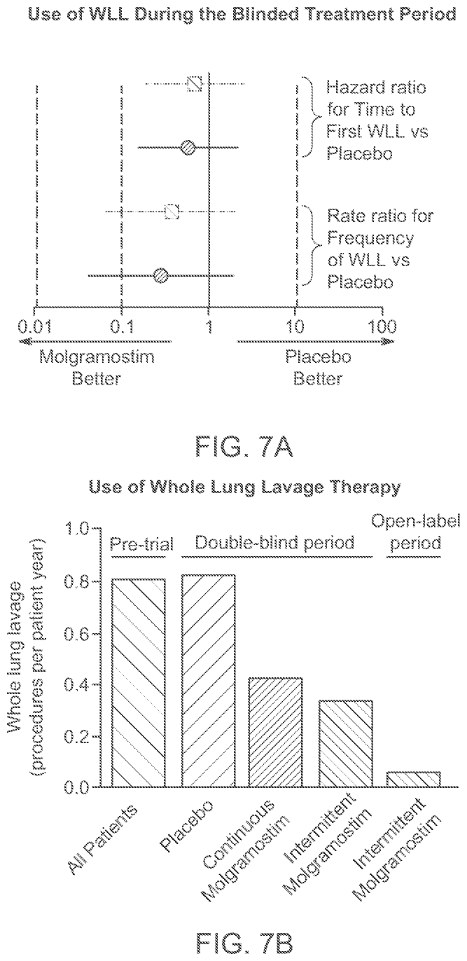

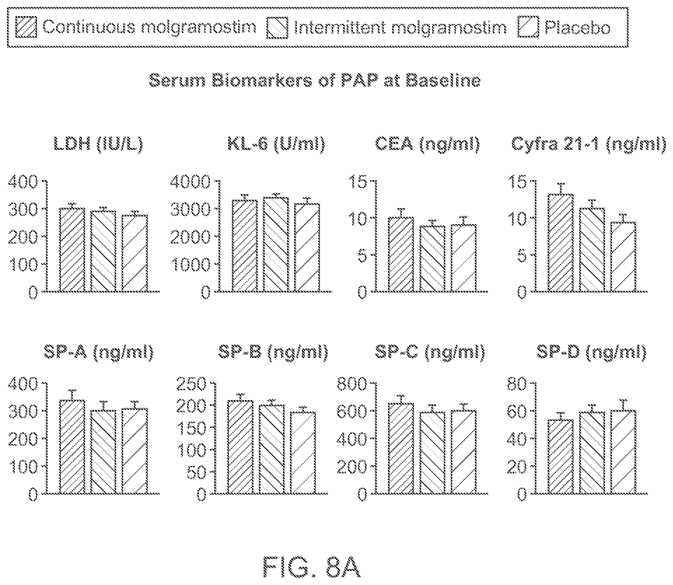

The use of whole-lung lavage therapy in the 2 years preceding enrollment was not significantly different among the three groups (FIG. 6). As compared with the rate of use of whole-lung lavage before enrollment (0.80 procedures per patient year), the rate remained similar in the placebo group after randomization (0.82 procedures per patient-year) but decreased in the continuous molgramostim group during the blinded intervention period (to 0.42 procedures per patient year) and decreased further during the open-label treatment-extension period (to 0.06 procedures per patient-year) (FIG. 7A). The time from baseline to the first use of whole-lung lavage therapy was not significantly different between patients receiving continuous molgramostim and those receiving placebo (FIG. 7B).

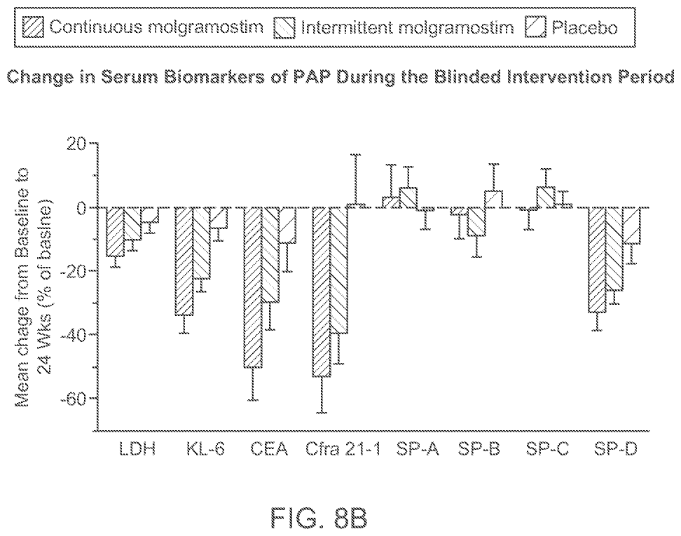

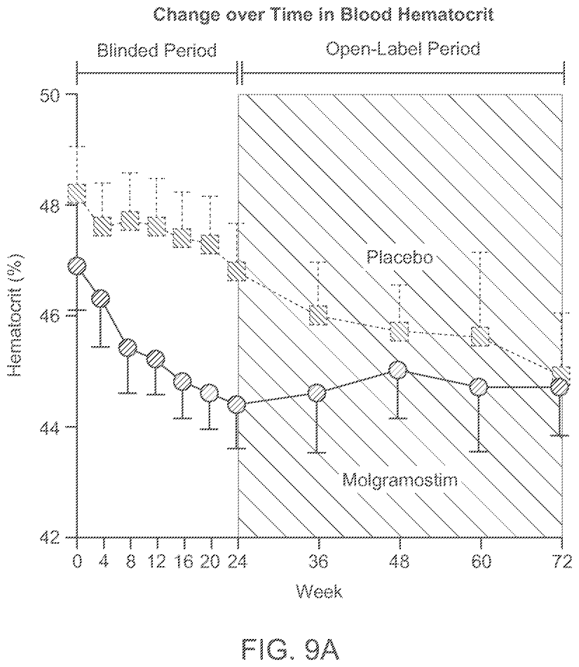

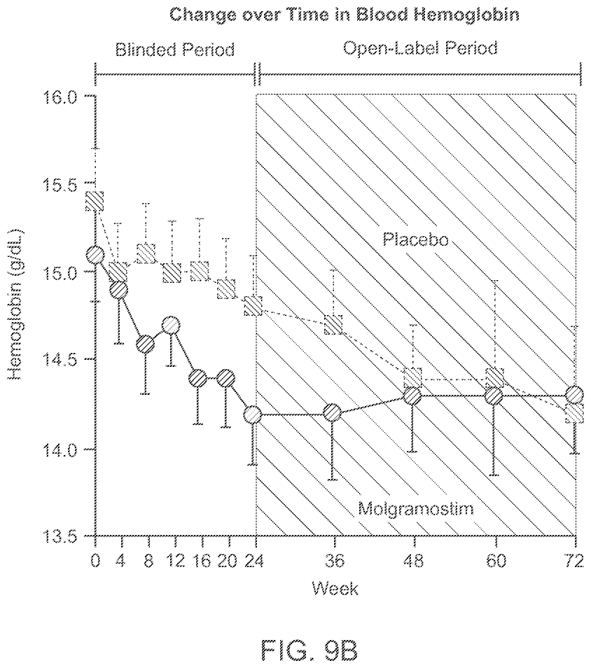

At week 24, the mean change from baseline in the ground-glass opacification score, reflecting the reduction in alveolar surfactant burden, was −3.6 points among patients receiving continuous molgramostim and −1.1 points among those receiving placebo (estimated treatment difference, −2.5 points; 95% CI, −3.7 to −1.2 by comparison of least-squares means) (Table 2). Improvements in levels of serum aPAP biomarkers among patients receiving continuous molgramostim and among those receiving placebo are shown in FIG. 8 and tab9.

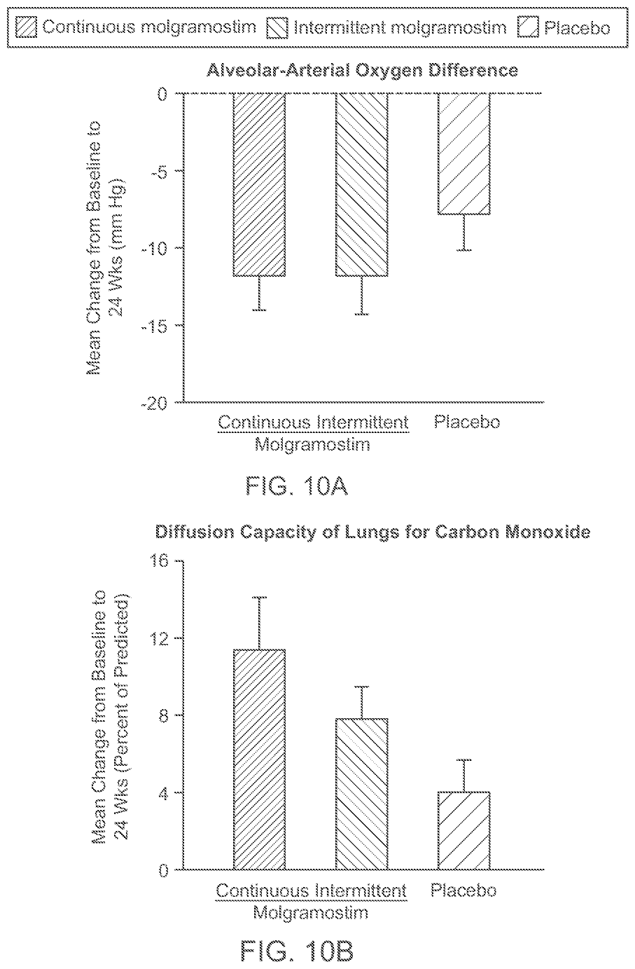

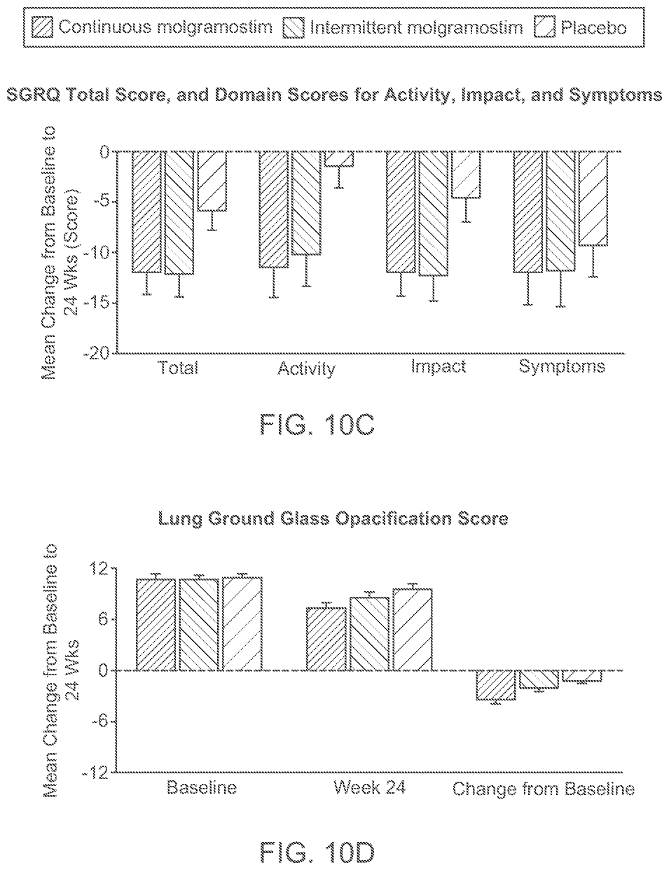

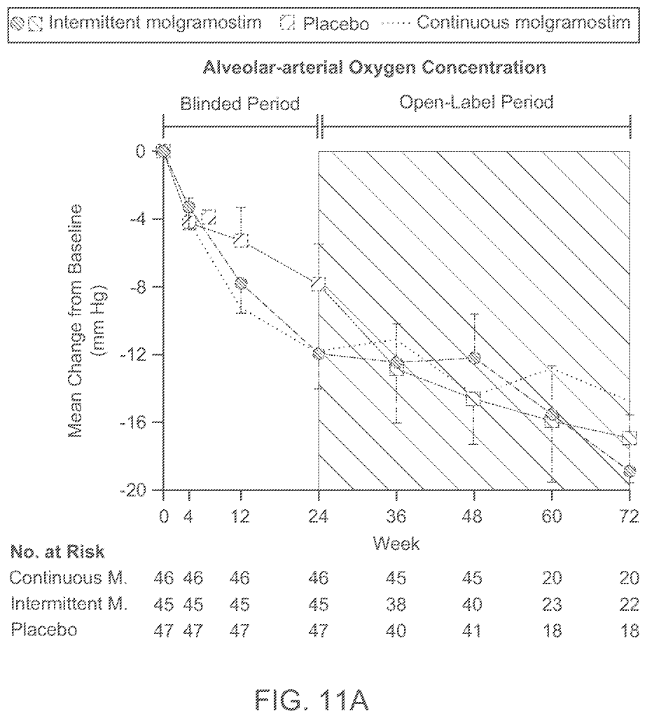

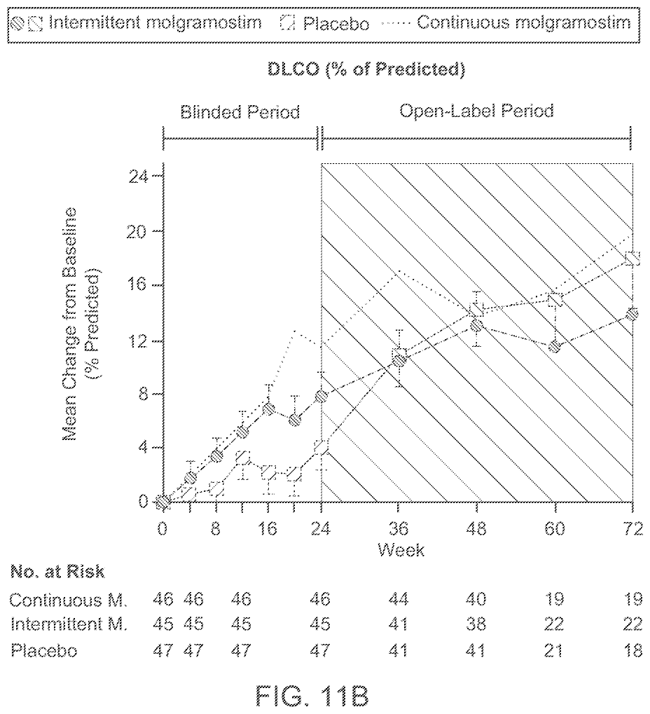

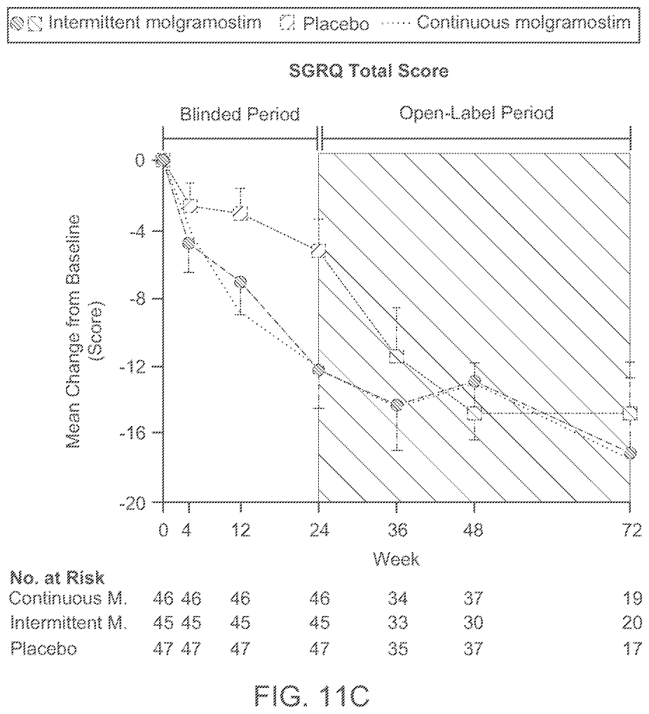

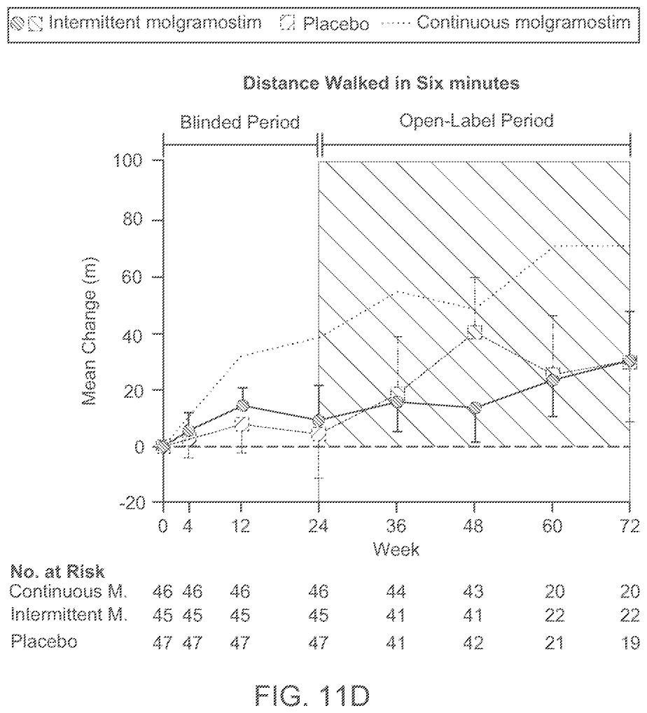

Because previous uncontrolled studies had evaluated intermittent GM-CSP administration in patients with aPAP, an intermittent-molgramostim group was included in this trial. The mean change from baseline in the A-aDO₂ at week 24 was not significantly different between the intermittent-molgramostim group and the placebo group when analyzed with the use of the revised full analysis set (−10.3 mm Hg vs. −6.6 mm Hg; estimated treatment difference, −3.8 mm Hg; P=0.18 by comparison of least-squares means). For most outcomes, the difference between the continuous-molgramostim group and the placebo group was greater than the difference between the intermittent-molgramostim group and the placebo group (FIGS. 10 and 11). The rate of use of whole-lung lavage during the blinded intervention period in the intermittent molgramostim group is shown in FIG. 7B.

During the open-label treatment-extension period, for which there was no control group, improvements in the A-aDO₂, DL_{CO}, SGRQ total score, and distance covered on the 6-minute walk test were observed among patients who had received continuous molgramostim, intermittent molgramostim, or placebo during the blinded intervention period (FIG. 11). The rate of use of whole-lung lavage during the open-label treatment-extension period is shown in FIG. 7B.

No deaths occurred during the trial. The percentages of patients with adverse events and serious adverse events were similar in the three groups during the blinded intervention period, except for the percentage of patients with chest pain which was 22% in the continuous-molgramostim group as compared with 4% in the intermittent-molgramostim group and 2% in the placebo group. No treatment-related serious adverse events were reported during the open-label treatment-extension period. The serum molgramostim (GMCSF) autoantibody titer was similar in the three groups at baseline and did not differ markedly during the blinded intervention period.

TABLE 2
Effects of Continuous Molgramostim in the Primary End Point and Selected Secondary End Points.*

| | | | | | Estimated |
|---|---|---|---|---|---|
| | Value at Week 24 | | Change from Baseline | | Difference |
| | Continuous | | Continuous | | Continuous |
| | Molgram- | | Molgram- | | Molgramostim |
| | ostim | Placebo | ostim | Placebo | vs. Placebo |
| Variable | (N = 46) | (N = 47) | (N = 46) | (N = 47) | (95% CI)† |
| Pulmonary gas transfer | | | | | |
| A-aDO₂ -mm Hg‡ | 26.4 ± 13.7 | 31.6 ± 12.7 | −11.9 ± 14.9 | −7.0 ± 11.4 | −6.2 |
| | | | | | (−11.7 to −0.8) |
| DL_{CO} - % of predicted value | 63.3 ± 22.5 | 53.6 ± 15.5 | 11.5 ± 17.4 | 4.0 ± 11.0 | 7.8 |
| | | | | | (2.3 to 13.3) |
| Radiologic evaluation | | | | | |
| of the lungs | | | | | |
| Ground glass opacification | 7.5 ± 3.6 | 10.0 ± 3.5 | −3.4 ± 3.8 | −1.2 ± 2.6 | −2.5 |
| score - points | | | | | (−3.7 to −1.2) |
| Functional health status | | | | | |
| SGRQ total score - points | 35.1 ± 21.3 | 38.9 ± 23.7 | −12.1 ± 14.3 | −5.2 ± 13.0 | −7.4 |
| | | | | | (−13.1 to −1.6) |
| Distance on 6-minute | 450 ± 135 | 451 ± 145 | 38.7 ± 95.6 | 4.3 ± 109.0 | 24.6 |
| walk test - m | | | | | (−15.3 to 64.4) |

*Plus-minus values are empirical means ± SD. Some values in the table differ from the values reported in the text for the same outcomes; the values in the text are least-square means. Details regarding the results for other end points are provided in Table S3.
†Between group differences is the change from baseline are presented as least-squares means and 95% confidence intervals (CIs) and were evaluated with use of an analysis of covariance model (with all trail groups included in the same model) that included trial group, status with regard to whole-lung lavage within 2 months before baseline (receipt vs. nonreceipt), and geographic region (Japan vs. other countries) as factors and baseline values as covariance.
‡The primary end point was the change from baseline in the A-aDO₂ data for four patients (one in each molgramostim group and two in the placebo group) were treated as missing data and were replaced by means of multiple imputation.

This trial showed that, among patients with aPAP, treatment with inhaled molgramostim for 24 weeks had beneficial effects, including greater improvement than placebo in measures of pulmonary gas transfer (A-aDO₂ and DL_{CO}), functional health status (SGRQ scores), and pathologic features (ground-glass opacification score and serum biomarker levels); however, no significant difference was observed in the distance covered on the 6-minute walk test or the use of whole-lung lavage therapy. Molgramostim was not associated with more frequent adverse effects, other than chest pain. The observation of synchronous greater improvement across multiple outcomes that reflect physiological, clinical, radiologic, and biochemical disease manifestations provides strong support for a treatment effect of molgramostim in patients with aPAP. Further support comes from the consistent trend toward greater efficacy when molgramostim was administered continuously rather than intermittently. Our data regarding SGRQ total scores show an improvement in health status among patients with aPAP in a controlled trial.

Daily administration of inhaled molgramostim led to greater improvement than placebo in outcomes that reflect physiological, radiologic, biochemical, and clinical manifestations of aPAP and was more beneficial when administered continuously than on alternating weeks.

### Example 2: Quantification of Anti-Molgramostim/Human GM-CSF Antibodies in Patient Serum

The potential development of anti-drug antibodies directed against molgramostim (recombinant human GM-CSP) was evaluated by measuring the titer of antiGM-CSF antibodies in serum from study patients using a homogeneous electrochemiluminescence-based bridging assay. Briefly, molgramostim was labeled with biotin and mixed with serum to permit formation of immune complexes between the biotinylated molgramostim and anti-GM-CSP antibodies present in serum. Immune complexes were captured on streptavidin-coated assay plates and antibodies were detected using Ru2+-containing Sulfo-tag™M. Importantly, antibodies against molgramostim and endogenous GM-CSP are both detected by this assay and cannot be distinguished. The methods were validated prior to analysis of study samples. In each run, high concentrated controls, low concentrated controls and negative controls were included. During the validation a plate-specific floating screening cut point was determined. Samples that were above the cut point in a screening assay were confirmed positive by inhibiting the signal with high levels of rhGMCSF. Samples showing over 40.1% inhibition in the confirmatory assay were scored positive and semiquantified by titration by analysis in serial 1:2 dilution(s). The highest titer equal to or above the floating cut point was reported.

### Example 3: Quantification of the GM-CSF-Neutralizing Capacity of Patient-Derived Serum

The GM-CSP neutralizing capacity of patient serum (caused by GM-CSP autoantibodies and or antimolgramostim antibodies) was measured using a cell proliferation assay using cultured TP-1 cells (DSMZ no. ACC 334), which normally proliferate in response to stimulation by GM-CSP.3 In this assay, neutralizing antibodies reduce GM-CSP-mediated growth stimulation. Briefly, cell growth was measured by spectrophotometric detection of formazan, which is produced by cells from 3-(4,5-dimethylthiazol-2-yl)-5-(3-carboxymethoxyphenyl)-2-(4-sulfophenyl)-2H-tetrazolium (MTS) in direct correlation to the number of cells present and was measured using a commercially available MTS kit (Promega G3581).

### Example 4: Evaluation of Chest CT Scans

Chest CT scans were evaluated by a visual scoring method as previously described. Briefly, the extent of ground glass opacification (GGO) of the lung parenchyma, a measure of surfactant accumulation in aPAP, was evaluated remotely by two blinded, independent radiologists who utilized dedicated software (Medidata) for the evaluation and recorded results directly into an electronic case report form. The area of lung parenchyma affected by GGO was measured using a zonal sampling of CT scan images including three transverse plane images representing regions of the upper lung Gust above the aortic arch), middle lung (at the main carina), and lower lung (at the bifurcation of the lingular and lower lobe bronchi). The area of GGO, which corresponds to the area of involvement according to the following scoring system: 0=no GGO, 1=less than 5% GGO, 2=5-24% GGO, 3=25-49% GGO, 4=50-74% GGO, 5=75% or more GGO. Both reviewers assigned a zonal HRCT GGO score for each region and the final GGO score was calculated as the average of all three regions. After completing the GGO score assessment, the reviewers determined if the extent of GGO at week 24 had changed compared to baseline. If reviewers did not agree on the extent of GGO when comparing images, an adjudicator reviewed the change assessment and made a determination without knowing the results of either Reader 1 or Reader 2. The radiologists also noted any adverse findings.

### Example 5: Arterial Blood Gas Measurement

Briefly, patients were placed in a supine position for 10 minutes prior to specimen collection. The analysis at Baseline was planned to be conducted with the patient breathing room air when possible. However, if the patient could not tolerate temporary discontinuation of supplemental oxygen during blood gas sampling at Baseline, blood gas sampling at subsequent visits should be conducted using the same oxygen flow rate as was used at Baseline. Blood specimens for arterial blood gas analysis at Baseline (Visit 2) and week 24 (Visit 8) were required to be obtained by arterial puncture. Use of capillary blood specimens was permitted for other times (Visit 1, 3, 5, and follow-up visits) for sites experienced in use of capillary specimens for blood gas analysis. The sample was analysed in accordance with local laboratory practices.

The following variables were measured: PaO₂ and PaCO₂. The A-aDO₂ was calculated based on PaO₂, PaCO₂ using a following equation:

$$Aa Gradient = ( {F}_{i} {O}_{2} ( {P}_{atm} - {P}_{{H}_{2} O} ) - \frac{{P}_{a} {CO}_{2}}{0.8} ) - {P}_{a} {O}_{2}$$

where Aa Gradient is the alveolar-arterial difference in oxygen concentration (A-aDO2), FiO2 is fraction of inspired oxygen, Pₐₜₘ is ambient atmospheric pressure, P_{H2O} is saturated vapour pressure of water, PₐCO₂ is arterial partial pressure of carbon dioxide, and PₐO₂ is arterial partial pressure of oxygen. The atmospheric pressure was determined at the time each test was performed. The FiO₂ was assumed to be 0.21, and the P_{H2O} was assumed to be 47 mm Hg (the value at normal body temperature).

### Example 6: Pulmonary Function Testing

Standardized pulmonary function testing was performed according to guidelines established by the American Thoracic Society/European Respiratory Society (ATS/ERS) Task Force by laboratory personnel with documented training in lung function testing. Atmospheric temperature and pressure were measured in parallel with pulmonary function testing. The lung function variables measured included forced expiratory volume in 1 second (FEV1), forced vital capacity (FVC), vital capacity (VC) and the diffusion capacity for carbon monoxide (DL_{CO}); all variables were expressed as a percentage of the predicted value—i.e., FEV1%, PVC%, VC %, and DL_{CO}%. DL_{CO} was adjusted for hemoglobin concentration.

During the double-blind treatment period, FEV1%, FVC%, and VC % were measured using a FlowScreen spirometer (eResearchTechnology GmbH, Estenfeld, Germany) were read centrally. DL_{CO} was performed using local equipment and results were read centrally. During the open label treatment period, spirometry and DL_{CO} were measured using local equipment.

### Example 7: Six-Minute Walk Test

The 6MWT was performed according to guidelines established by the ATS/ERS Task Force 13 by technicians with documented training and experience of performing the 6MWT. Whenever possible, the 6MWT was conducted with the patient breathing room air. If the patient required oxygen supplementation at rest, an oxygen titration procedure was followed as part of the 6MWT at the Screening Visit in order to determine the amount of oxygen supplementation required for the patient to complete the test. The same flow of oxygen should then be used at the patient's subsequent tests in the trial, if possible.

### Example 8: Disease Severity Score

The aPAP disease severity score (DSS) developed by Inoue (Inoue et al. American journal of respiratory and critical care medicine 2008; 177:752-62, which is incorporated herein by reference for all purposes) is based on the presence of PAP-related symptoms and the degree of reduction in arterial oxygen concentration (PaO2) determined with the individual breathing room air in the supine position. The DSS uses a 5-point scale to measure aPAP disease severity as follows: 1=no symptoms and PaO2 ˜70 mmHg, 2=symptomatic and PaO2˜70 mmHg, 3=60 mmHg˜PaO2<70 mmHg, 4=50 mmHg˜PaO2<60 mmHg, 5=PaO2<50 mmHg.

### Example 9: Functional Health Status

Patients were asked to complete the St Georges Respiratory Questionnaire (SGRQ) 15 (translated into the local language for each country) using 4-week recall. The SGRQ was originally designed to measure impact on health status in patients with obstructive airway disease but most of the questions were considered relevant for the evaluation of patients with aPAP.

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations, or methods, or any combination of such changes and modifications of use of the invention, may be made without departing from the spirit and scope thereof.

All references (patent and non-patent) cited above are incorporated by reference into this patent application. The discussion of those references is intended merely to summarize the assertions made by their authors. No admission is made that any reference (or a portion of any reference) is relevant prior art (or prior art at all). Applicant reserves the right to challenge the accuracy and pertinence of the cited references.

1. A method for the treatment of moderate-severe pulmonary alveolar proteinosis (aPAP) comprising administering a formulation comprising 300 μg molgramostim once daily by inhalation to a patient in need thereof.

2. The method of claim 1, wherein the formulation further comprises a stabilizer.

3. The method of claim 2, wherein the stabilizer is recombinant human albumin.

4. The method of claim 1, wherein the molgramostim is administered continuously.

5. The method of claim 1, wherein the molgramostim is administered continuously intermittently.

6. The method of claim 1, wherein the formulation is delivered via a high-efficiency nebulizer.
